(19) United States
(12) Patent Application Publication
(10) Pub. No.: US 2012/0277852 A1
(43) Pub. Date: Nov. 1, 2012

(54) COATING COMPOSITIONS, METHODS AND COATED DEVICES

(75) Inventors: Anita Shukla, Houston, TX (US); Paula T. Hammond, Newton, MA (US)

(73) Assignee: MASSACHUSETTS INSTITUTE OF TECHNOLOGY, Cambridge, MA (US)

(21) Appl. No.: 13/459,069

(22) Filed: Apr. 27, 2012

Related U.S. Application Data
(60) Provisional application No. 61/479,525, filed on Apr. 27, 2011.

(51) Int. Cl. A61F 2/82; A61F 2/24; B05D 1/02

(52) U.S. Cl. 623/1.43; 427/2.1; 623/1.42; 623/2.42

(57) ABSTRACT

In various embodiments, a coated device comprises: a substrate; a film coating at least part of the substrate, which film comprises a multilayer unit comprising a first layer and a second layer associated with one another via a hydrogen bond, wherein the first layer comprises a first natural polymeric material and a hydrogen bond donor and wherein the second layer comprises a second natural polymeric material and a hydrogen bond acceptor; and an agent for delivery associated with the coated device. In various embodiments, a coated device comprises: a substrate; a film coating at least part of the substrate, which film comprises a multilayer unit comprising a tetralayer with alternating layers of opposite charge; and an agent for delivery associated with the coated device.

### RELATED REFERENCES

This application claims priority to U.S. provisional patent application Ser. No. 61/479,525, filed Apr. 27, 2011, the entire contents of which are herein incorporated by reference.

### GOVERNMENT SUPPORT

This invention was made with government support under Grant No. W911NF-07-D-0004 awarded by the Army Research Office. The government has certain rights in this invention.

### BACKGROUND

It is often desirable to delivery one or more agents such as drugs from medical devices that are used in association with a body. For example, such devices, can create infection, inflammation or other risks for subjects. Additionally, such devices are by their nature localized in or on a body, and can act as useful systems for local administration of therapeutic or other agents.

### SUMMARY

The present disclosure provides, among other things, a coated device comprising: a substrate; a film coating at least part of the substrate, which film comprises a multilayer unit comprising a first layer and a second layer adjacent to the first layer, wherein the first layer comprises a first polymeric material and at least first interacting moiety, wherein the second layer comprises a second polymeric material and at least second interacting moiety, and wherein the interacting moieties on adjacent layers interact with one another so that the adjacent layers are associated with each other into the film; and an agent for delivery associated with the coated device, such that decomposition of one or more layers of the film results in release of the agent.

In some embodiments, a coated device comprising: a substrate; a film coating at least part of the substrate, which film comprises a multilayer unit comprising a first layer and a second layer associated with one another via a hydrogen bond, wherein the first layer comprises a first natural polymeric material and a hydrogen bond donor and wherein the second layer comprises a second natural polymeric material and a hydrogen bond acceptor; and an agent for delivery associated with the coated device such that, decomposition of one or more layers of the film results in release of the agent.

In some embodiments, a coated device comprising: a substrate; a film coating at least part of the substrate, which film comprises a multilayer unit comprising a tetralayer with alternating layers of opposite charge; and an agent for delivery associated with the coated device such that, decomposition of one or more layers of the film results in release of the agent.

In some embodiments, the present invention encompasses the recognition that it is desirable and beneficial in some cases to create and/or utilize an LBL film comprising an agent to be delivered where at least one layer consists of the agent to be delivered. That is, the agent itself is used to make the layer.

In some embodiments, the present invention encompasses the further recognition that many or most traditional approaches to LBL films utilize and/or require electrostatic intra-layer interactions. The present invention provides the insight that at least some potential layer materials, including potential agents for delivery that could otherwise be utilized as layer materials do not and/or cannot carry sufficient charge to mediate stable electrostatic interactions.

In some embodiments, the present invention provides and/or encompasses LBL films in which at least two individual layers within the film interact and/or associate through interactions other than or more than electrostatic interactions. In addition to electrostatic interactions or alternatively, at least two individual layers within the film interact and/or associate through non-covalent interactions selected from the group consisting of hydrogen bonding, affinity interactions, metal coordination, physical adsorption, host-guest interactions, hydrophobic interactions, pi stacking interactions, hydrogen bonding interactions, van der Waals interactions, magnetic interactions, dipole-dipole interactions and combinations thereof. In some particular such embodiments, at least one of the two individual interacting layers is or comprises agent to be delivered. In some such embodiments, at least one of the two individual interacting layers consisting of agent to be delivered.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Associated”: As used herein, the term “associated” typically refers to two or more moieties connected with one another, either directly or indirectly (e.g., via one or more additional moieties that serve as a linking agent), to form a structure that is sufficiently stable so that the moieties remain connected under the conditions in which the structure is used, e.g., physiological conditions. In some embodiments, associated moieties are attached to one another by one or more covalent bonds. In some embodiments, associated moieties are attached to one another by a mechanism that involves specific (but non-covalent) binding (e.g. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Hydrolytically degradable”: As used herein, “hydrolytically degradable” polymers are polymers that degrade fully in the sole presence of water. In preferred embodiments, the polymers and hydrolytic degradation byproducts are biocompatible. As used herein, the term “non-hydrolytically degradable” refers to polymers that do not fully degrade in the sole presence of water.

“Nucleic acid”: The term “nucleic acid” as used herein, refers to a polymer of nucleotides. Deoxyribonucleic acids (DNA) or ribonucleic acids (RNA) and polymers thereof in either single- or double-stranded form are exemplary polynucleotides. Unless specifically limited, the term encompasses nucleic acid molecules containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. In some embodiments, a polynucleotide sequence of relatively shorter length (e.g., no more than 50 nucleotides, preferably no more than 30 nucleotides, and more preferably no more than 15-20 nucleotides) is typically referred to as an “oligonucleotide.”

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polyelectrolyte”: The term “polyelectrolyte”, as used herein, refers to a polymer which under some set of conditions (e.g., physiological conditions) has a net positive or negative charge. Polyelectrolytes includes polycations and polyanions. Polycations have a net positive charge and polyanions have a net negative charge. The net charge of a given polyelectrolyte may depend on the surrounding chemical conditions, e.g., on the pH.

“Polypeptide”: The term “polypeptide” as used herein, refers to a string of at least three amino acids linked together by peptide bonds. Polypeptides such as proteins may contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”: The term “polysaccharide” refers to a polymer of sugars. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Substantial” or “substantive”: As used herein, the terms “substantial” or “substantive” and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Treating”: As used herein, the term refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### BRIEF DESCRIPTION OF DRAWING

FIG. 1 illustrates spray layer-by-layer assembly for porous substrates. Each airbrush aerosolizes and sprays film components or the rinse solution at the substrate; a vacuum is applied to pull solutions through the substrate. For the vancomycin LbL films, 1=poly 2, 2 and 4=dextran sulfate, and 3=vancomycin.

FIG. 2 illustrates typical SEM micrographs of uncoated and (poly 2/dextran sulfate/vancomycin/dextran sulfate)ₙ spray LbL coated commercial gelatin sponges. Scale bar=500 μm and 50 μm for top and bottom row micrographs for both plan-view and cross-section images (except 60 tetralayer cross-section top row, where scale bar=200 μm), respectively.

FIG. 3 illustrates exemplary absorbency ratio of phosphate buffered saline by film coated compared to uncoated gelatin sponges

FIG. 4 illustrates typical Vancomycin release profiles from gelatin sponges coated with (poly 2/dextran sulfate/vancomycin/dextran sulfate)ₙ where n=60 and 120. A.) Drug release expressed in μg of vancomycin per mg of sponge. B.) Drug release expressed in μg of vancomycin per sponge projected in-plane area (cm²).

FIG. 5 illustrate typical Normalized vancomycin release profiles. A.) Complete release from gelatin sponges and flat substrates coated with (poly 2/dextran sulfate/vancomycin/dextran sulfate)₆₀ spray LbL films and vancomycin-soaked sponges (no film). B.) Data shown in (A.) up to 52 hours of release. C.) Complete release from gelatin sponges and flat substrates coated with (poly 2/dextran sulfate/vancomycin/dextran sulfate)₁₂₀ spray LbL films and vancomycin-soaked sponges (no film). D.) Data shown in (C.) up to 77 hours of release.

FIG. 6 illustrates an exemplary study of Staphylococcus aureus growth inhibition. A.) Normalized S. aureus density upon exposure to dilutions of film release solutions from LbL coated gelatin sponges and a control of non-film released vancomycin (dilution 1=2.3, 2.3, and 1.9 μg/mL for the vancomycin control, n=60, and n=120, respectively; each subsequent dilution is half the concentration of the previous dilution). B.) Agar coated with S. aureus exposed to 60 tetralayer LbL film coated gelatin sponges(i and ii), an uncoated piece of sponge (iii), and a 30 μg vancomycin control disc (iv). Sample (i) is the top two-thirds of the coated sponge, while sample (ii) is the bottom one-third.

FIG. 7 shows typical Vancomycin release from (poly 2/dextran sulfate/vancomycin/dextran sulfate)₁₂₀ coated gelatin sponges. A.) Release from three individual samples is shown; the average of these three samples leads to the results shown in FIGS. 5C and 5D. B.) The total vancomycin released for the last three time points of significant release showing that each individual sample releases a significant quantity of vancomycin through 150 hours.

FIG. 8 illustrates exemplary results of (Thrombin/tannic acid)ₙ growth and dissolution. A.) QCM film growth for (thrombin/tannic acid)ₙ and (mannitol/tannic acid)ₙ on a BPEI monolayer (B=start of BPEI, arrow=start of thrombin, triangle=start of tannic acid; a 5 minute wash in PBS preeceds the start of each deposition step). B.) Average thickness of sprayed (thrombin/tannic acid)ₙ films and change in thickness per bilayer from 0 to 10, 10 to 25, and 25 to 50 bilayers. C.) Sprayed (thrombin/tannic acid)ₙ film dissolution in 0.01 M PBS at 37° C.

FIG. 9 illustrates exemplary results of a sprayed (thrombin/tannic acid)ₙ morphology. A.) Atomic force microscope images of films on flat substrates (10 μm×10 μm; zₘₐₓ=360 nm, 380 nm, and 440 nm, and RMS roughness=46.3±3.7 nm, 51.9±4.2 nm, 66.8±11.5 nm for n=10, 25, and 50, respectively). B.) Plan-view scanning electron microscope images of film coated gelatin sponges for n=0, 10, 25, and 50 (scale bar =200 μm).

FIG. 10 illustrates hemostatic activity of an exemplary film coated gelatin sponge. A.) In vitro sponge activity. B.) Sprayed film thickness on flat substrates and in vitro activity of coated sponge. C.) Porcine spleen bleeding model. B.) Time to hemostasis following sample application (controls were sponges with a monolayer coating of BPEI).

### DETAILED DESCRIPTION OF CERTAIN EMBODIMENTS

In various embodiments, compositions and methods for constructing an LBL film associated with one or more agents for delivery to coat a substrate are disclosed. Provided LBL films and methods can be used to coat a substrate for controlled delivery of one or more agents.

### LBL Films

LBL films may have various film architecture, film materials, film thickness, surface chemistry, and/or incorporation of agents according to the design and application of coated devices.

In general, LBL films comprise multiple layers. In many embodiments, LBL films are comprised of multilayer units; each unit comprising individual layers. In accordance with the present disclosure, individual layers in an LBL film interact with one another. In particular, a layer in an LBL film comprises an interacting moiety, which interact with that from an adjacent layer, so that a first layer associates with a second layer adjacent to the first layer, each contains at least one interacting moiety.

In some embodiments, adjacent layers are associated with one another via non-covalent interactions. Exemplary non-covalent interactions include, but are not limited to, hydrogen bonding, affinity interactions, metal coordination, physical adsorption, host-guest interactions, hydrophobic interactions, pi stacking interactions, hydrogen bonding interactions, van der Waals interactions, magnetic interactions, dipole-dipole interactions and combinations thereof.

In some embodiments, an interacting moiety is a charge, positive or negative. In some embodiments, an interacting moiety is a hydrogen bond donor or acceptor. In some embodiments, an interacting moiety is a complementary moiety for specific binding such as avidin/biotin. In various embodiments, more than one interactions can be involve in the association of two adjacent layers. For example, an electrostatic interaction can be a primary interaction; a hydrogen bonding interaction can be a secondary interaction between the two layers.

LBL films may be comprised of multilayer units with alternating layers of opposite charge, such as alternating anionic and cationic layers.

In some embodiments, the present invention provides the insight that at least some potential layer materials, including potential agents for delivery that could otherwise be utilized as layer materials do not and/or cannot carry sufficient charge to mediate stable electrostatic interactions. In addition to electrostatic interaction or alternatively, they can be associated via non-electrostatic interaction in a coated device in accordance with the present invention.

According to the present disclosure, LBL films may be comprised of one or more multilayer units. In some embodiments, an LBL film include a plurality of a single unit (e.g., a bilayer unit, a tetralayer unit, etc.). In some embodiments, an LBL film is a composite that include more than one units. For example, more than one units can have be different in film materials (e.g., polymers), film architecture (e.g., bilayers, tetralayer, etc.), film thickness, and/or agents that are associated with one of the units. In some embodiments, an LBL film is a composite that include more than one bilayer units, more than one tetralayer units, or any combination thereof. In some embodiments, an LBL film is a composite that include a plurality of a single bilayer unit and a plurality of a single tetralayer unit.

In some embodiments, the number of a multilayer unit is 3, 5, 10, 20, 30, 40, 50, 60, 70, 80, 90, 100, 150, 200, 300, 400 or even 500.

LBL films may have various thickness depending on methods of fabricating and applications. In some embodiments, an LBL film has an average thickness in a range of about 1 nm and about 100 μm. In some embodiments, an LBL film has an average thickness in a range of about 1 μm and about 50 μm. In some embodiments, an LBL film has an average thickness in a range of about 2 μm and about 5 μm. In some embodiments, the average thickness of an LBL film is or more than about 1 nm, about 100 nm, about 500 nm, about 1 μm, about 2 μm, about 3 μm, about 4 μm, about 5 μm, about 10 μm, bout 20 μm, about 50 μm, about 100 μm. In some embodiments, an LBL film has an average thickness in a range of any two values above.

An individual layer of an LBL film can contain a polymeric material. In some embodiments, a polymer is degradable or non-degradable. In some embodiments, a polymer is natural or synthetic.

In some embodiments, a polymer is a polyelectrolyte.

In some embodiment, a polymer is a polypeptide. In some embodiments, a polymer has a relatively small molecule weight. In some embodiments, a polymer is an agent for delivery. For example, model agents for delivery such as thrombin and vancomycin are demonstrated in Examples 1 and 2 below.

LBL films can be decomposable. In many embodiments, a polymer of an individual layer includes a degradable polyelectrolyte. In some embodiments, decomposition of LBL films is characterized by substantially sequential degradation of at least a portion of the polyelectrolyte layers that make up LBL films. Degradation may be at least partially hydrolytic, at least partially enzymatic, at least partially thermal, and/or at least partially photolytic. Degradable polyelectrolytes and their degradation byproducts may be biocompatible so as to make LBL films amenable to use in vivo.

Degradable polyelectrolytes can be used in an LBL film disclosed herein, including, but not limited to, hydrolytically degradable, biodegradable, thermally degradable, and photolytically degradable polyelectrolytes. Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. Of course, co-polymers, mixtures, and adducts of these polymers may also be employed.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself. Cationic polyelectrolytes may be degradable polymers with cationic groups distributed along the polymer backbone. Cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself.

For example, a range of hydrolytically degradable amine containing polyesters bearing cationic side chains have been developed. Examples of these polyesters include poly(L-lactide-co-L-lysine), poly(serine ester), poly(4-hydroxy-L-proline ester), and poly[α-(4-aminobutyl)-L-glycolic acid].

In addition, poly(β-amino ester)s, prepared from the conjugate addition of primary or secondary amines to diacrylates, are suitable for use. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, a polymer can have a formula below:

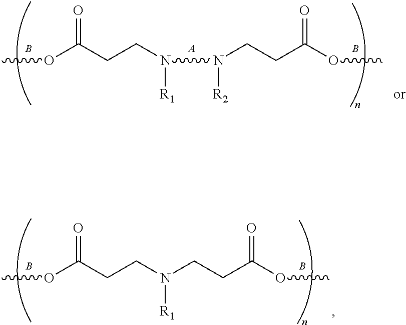

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

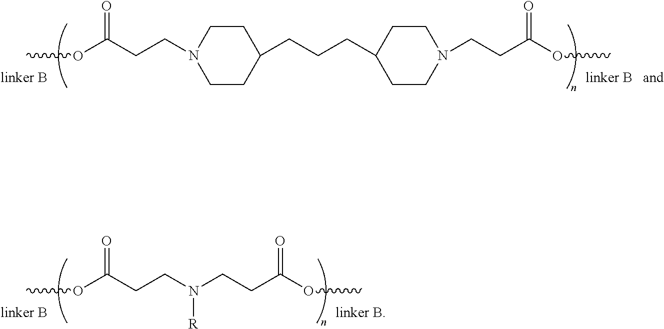

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units.

In some embodiments, a poly(β-amino ester)s are selected from the group consisting of

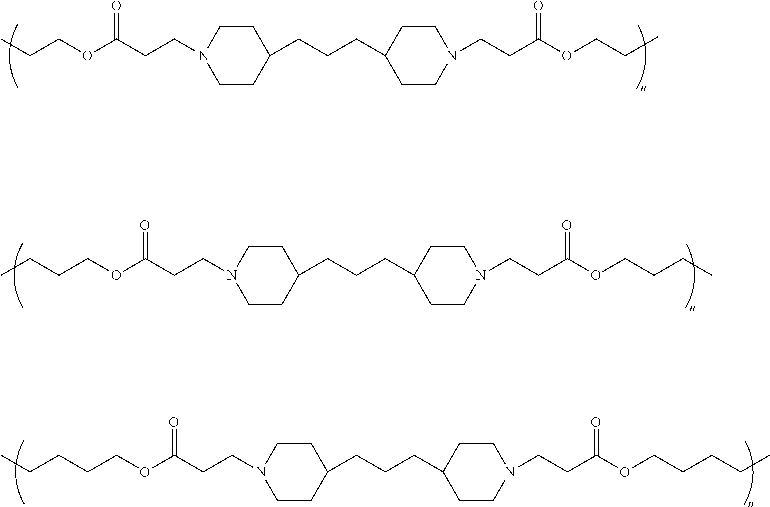

derivatives thereof, and combinations thereof.

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, an LBL film may be constructed by LbL deposition using dip coating in solutions of a first pH at which one layer is anionic and a second layer is cationic. If such an LBL film is put into a solution having a second different pH, then the first layer may be rendered cationic while the second layer is rendered anionic, thereby changing the charges on those layers.

The composition of degradable polyeletrolyte layers can be fine-tuned to adjust the degradation rate of each layer within the film, which is believe to impact the release rate of drugs. For example, the degradation rate of hydrolytically degradable polyelectrolyte layers can be decreased by associating hydrophobic polymers such as hydrocarbons and lipids with one or more of the layers. Alternatively, polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions.

In other embodiments, polyanionic and/or polycationic layers may include a mixture of degradable and non-degradable polyelectrolytes. Any non-degradable polyelectrolyte can be used. Exemplary non-degradable polyelectrolytes that could be used in thin films include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Alternatively or additionally, the degradation rate may be fine-tuned by associating or mixing non-biodegradable, yet biocompatible polymers with one or more of the polyanionic and/or polycationic layers. Suitable non-biodegradable, yet biocompatible polymers are well known in the art and include polystyrenes, certain polyesters, non-biodegradable polyurethanes, polyureas, poly(ethylene vinyl acetate), polypropylene, polymethacrylate, polyethylene, polycarbonates, and poly(ethylene oxide)s.

Polymers used herein in accordance with the present disclosure generally can be biologically derived or natural. Polymers that may be used include charged polysaccharides. In some embodiments, polysaccharides include glycosaminoglycans such as heparin, chondroitin, dermatan, hyaluronic acid, etc. (Some of these terms for glycoasminoglycans are often used interchangeably with the name of a sulfate form, e.g., heparan sulfate, chondroitin sulfate, etc. It is intended that such sulfate forms are included among a list of exemplary polymers used in accordance with the present invention.).

Additionally or alternatively, polymers can be a natural acid. For example, tannic acid is used in Example 2 serving as a layer of a bilayer.

LBL films may be exposed to a liquid medium (e.g., intracellular fluid, interstitial fluid, blood, intravitreal fluid, intraocular fluid, gastric fluids, etc.). In some embodiments, an LBL film comprises at least one polycationic layer that degrades and at least one polyanionic layer that delaminates sequentially. Releasable agents are thus gradually and controllably released from the LBL film. It will be appreciated that the roles of the layers of an LBL film can be reversed. In some embodiments, an LBL film comprises at least one polyanionic layer that degrades and at least one polycationic layer that delaminates sequentially. Alternatively, polycationic and polyanionic layers may both include degradable polyelectrolytes.

### Agents for Delivery

Coated devices utilized in accordance with the present invention can comprise one or more agents for delivery. In some embodiments, one or more agents are associated independently with a substrate, an LBL film coating the substrate, or both in a coated device.

In some embodiments, an agent can be associated with individual layers of an LBL film for incorporation, affording the opportunity for exquisite control of loading and release from the film. In certain embodiments, an agent is incorporated into an LBL film by serving as a layer.

In some embodiments, an agent for delivery is released when one or more layers of a LBL film are decomposed. Additionally or alternatively, an agent is release by diffusion.

In theory, any agents including, for example, therapeutic agents (e.g. antibiotics, NSAIDs, glaucoma medications, angiogenesis inhibitors, neuroprotective agents), cytotoxic agents, diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be associated with the LBL film disclosed herein to be released.

In some embodiments, compositions and methods in accordance with the present disclosure are particularly useful for hemostatic coating by releasing of one or more clotting factor. Exemplary clotting factors include, but are not limited to, Factors I-XIII (e.g., fibrinogen, thrombin, tissue factor), von Willebrand factor, fletcher factor, fitzgerald factor, fibronectin, antithrombin III, heparin cofactor II, protein C, protein S, protein Z, ZPI, plasminogen, alpha 2-antiplasmin, tPA, urokinase, PAI1, PAI2, cancer procoagulant, and fragments and variants thereof.

In some embodiments, agents for delivery utilized in accordance with the present disclosure are one or more therapeutic agents. Exemplary agents include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, a therapeutic agent to be delivered is an agent useful in combating inflammation and/or infection.

In some embodiments, a therapeutic agent is a small molecule and/or organic compound with pharmaceutical activity. In some embodiments, a therapeutic agent is a clinically-used drug. In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof.

An antibiotic used in accordance with the present disclosure may be bacteriocidial or bacteriostatic. Other anti-microbial agents may also be used in accordance with the present disclosure. For example, anti-viral agents, anti-protazoal agents, anti-parasitic agents, etc. may be of use.

In some embodiments, a therapeutic agent may be an anti-inflammatory agent. Anti-inflammatory agents may include corticosteroids (e.g., glucocorticoids), cycloplegics, non-steroidal anti-inflammatory drusg (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Additionally or alternatively, an agent having NSAID-like activity can be used. Suitable compounds having NSAID activity include, but are non-limited to, the non-selective COX inhibitors, selective COX-2 inhibitors, selective COX-1 inhibitors, and COX-LOX inhibitors, as well as pharmaceutically acceptable salts, isomers, enantiomers, polymorphic crystal forms including the amorphous form, co-crystals, derivatives, prodrugs thereof.

Those skilled in the art will recognize that this is an exemplary, not comprehensive, list of agents that can be released using compositions and methods in accordance with the present disclosure. In addition to a therapeutic agent or alternatively, various other agents may be associated with a coated device in accordance with the present disclosure.

### Substrates

A variety of materials can be used as a substrate for constructing LBL films. For example, a coated device in accordance with the present invention comprises one or more LBL films coated on at least one surface of a substrate.

In some embodiments, a material of a substrate is metals (e.g., gold, silver, platinum, and aluminum); metal-coated materials; metal oxides; and combinations thereof.

In some embodiments, a material of a substrate is plastics, ceramics, silicon, glasses, mica, graphite or combination thereof.

In some embodiments, a material of a substrate is a polymer. Exemplary polymers include, but are not limited to, polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof.

In some embodiments, a substrate may comprise more than one material to form a composite.

A substrate can be a medical device. Some embodiments of the present disclosure comprise various medical devices, such as sutures, bandages, clamps, valves, intracorporeal or extracorporeal devices (e.g., catheters), temporary or permanent implants, stents, vascular grafts, anastomotic devices, aneurysm repair devices, embolic devices, and implantable devices (e.g., orthopedic implants) and the like.

In some embodiments, a medical device is catheter. Catheters are widely used in medical applications, e.g., for intravenous, arterial, peritoneal, pleural, intrathecal, subdural, urological, synovial, gynecological, percutaneous, gastrointestinal, abscess drains, and subcutaneous applications. Intravenous infusions are used for introducing fluids, nutrition, blood or its products, and medications to patients. These catheters are placed for short-term, intermediate, and long-term usage. Types of catheters include standard IV, peripherally inserted central catheters (PICC)/midline, central venous catheters (CVC), angiographic catheters, guide catheters, feeding tubes, endoscopy catheters, Foley catheters, drainage catheters, and needles. Catheter complications include phlebitis, localized infection and thrombosis.

In some embodiments, medical devices are retractors or forceps, which is commonly used in surgery to position or move (e.g., manipulate) organs and tissues for better visualization, surgical approach, and placement of implants. Dentistry commonly uses forceps to position small tooth restorations (e.g., crowns, inlays, on lays, veneers, implants/implant abutments, etc.) and position gingival tissues in a variety of periodontal, oral surgical and endodontic procedures. The current existing dental device in this market sector is a sticky ended probe (Grabits™) that is disliked by dentists as it is non-sterile, cannot adhere to living tissue and is difficult to release from the implant it is adhered to.

In some embodiments, a medical device is an external fixator implant. External fixators are pins and wires inserted through the skin into bone for the purpose of healing bone fractures. These pins and wires are then connected externally with rods and clamps in order to provide rigidity and stability so the fractured bone can heal.

In some embodiments, a medical device is an intraluminal camera. One of the latest diagnostic advances is the use of miniaturized, untethered cameras to observe internal organs. Such cameras, the size of pills, may be ingested or injected and float downstream, sending images back to the medical observer.

In some embodiments, a medical device is a mechanical heart valve. There are two types of heart valve prostheses used for replacement of aortic and mitral valves. Mechanical valves commonly are metallic cages with a disc that opens at systole to allow blood to flow and closes at diastole to prevent backflow. These valves last indefinitely but require the daily administration of an anticoagulant drug to prevent thrombotic complications. The dose must be carefully regulated to prevent thrombus formation on one hand and internal hemorrhage on the other. The other type of valve is the tissue valve, sometimes isolated en bloc from porcine hearts and sometimes constructed from bovine pericardial tissue. These leaflet valves are more like natural valves and usually do not require anticoagulant drug administration. However, they are susceptible to degradation and have more finite life expectancies than do the mechanical valves. In certain embodiments, hemostatic LBL films as demonstrated in Example 2 may be particularly useful in accordance with the present disclosure to coat a heart valve.

In some embodiments, a medical device is a vascular stent. More than 70 coronary stents have been approved in Europe and over 20 stents are commercially available in the United States such as the Multi-Link Vision™ Coronary Stent System available commercially from Guidant Corporation (Indianapolis, Ind.), and the Driver™ Coronary Stent System or BeStent2™ available commercially from Medtronic, Inc. (Minneapolis, Minn.).

In some embodiments, a medical device an implantable sensor such as glucose sensors, cardiac function sensors (either on-lead or off) and neurological implants of various stripes.

In some embodiments, a medical device is an orthopedic implant. LBL films can be used in accordance with the present disclosure to coat orthopedic implants. Examples of orthopedic implants include without limitation total knee joints, total hip joints, ankle, elbow, wrist, and shoulder implants including those replacing or augmenting cartilage, long bone implants such as for fracture repair and external fixation of tibia, fibula, femur, radius, and ulna, spinal implants including fixation and fusion devices, maxillofacial implants including cranial bone fixation devices, artificial bone replacements, dental implants, orthopedic cements and glues comprised of polymers, resins, metals, alloys, plastics and combinations thereof, nails, screws, plates, fixator devices, wires and pins and the like that are used in such implants, and other orthopedic implant structures as would be known to those of ordinary skill in the art.

In some embodiments, medical devices are not intraocular lenses (IOLs).

### Methods and Uses

There are several advantages to LBL assembly techniques used to coat a substrate in accordance with the present disclosure, including mild aqueous processing conditions (which may allow preservation of biomolecule function); nanometer-scale conformal coating of surfaces; and the flexibility to coat objects of any size, shape or surface chemistry, leading to versatility in design options. According to the present disclosure, one or more LBL films can be assembled and/or deposited on a substrate to provide a coated device. In many embodiments, a coated device having one or more agents for delivery associated with it, such that decomposition of layers of LBL films results in release of the agents.

In various embodiments, LBL films can be different in film materials (e.g., polymers), film architecture (e.g., bilayers, tetralayer, etc.), film thickness, and/or agent association depending on methods and/or uses. In many embodiments, a coated device in accordance with the present disclosure is for medical use.

It will be appreciated that an inherently charged surface of a substrate can facilitate LbL assembly of an LBL film on the substrate. In addition, a range of methods are known in the art that can be used to charge the surface of a substrate, including but not limited to plasma processing, corona processing, flame processing, and chemical processing, e.g., etching, micro-contact printing, and chemical modification.

In some embodiments, substrate can be coated with a base layer. Additionally or alternatively, substrates can be primed with specific polyelectrolyte bilayers such as, but not limited to, LPEI/SPS, PDAC/SPS, PAH/SPS, LPEI/PAA, PDAC/PAA, and PAH/PAA bilayers, that form readily on weakly charged surfaces and occasionally on neutral surfaces. Exemplary polymers can be used as a primer layer include poly(styrene sulfonate) and poly(acrylic acid) and a polymer selected from linear poly(ethylene imine), poly(diallyl dimethyl ammonium chloride), and poly(allylamine hydrochloride). It will be appreciated that primer layers provide a uniform surface layer for further LBL assembly and are therefore particularly well suited to applications that require the deposition of a uniform thin film on a substrate that includes a range of materials on its surface, e.g., an implant or a complex tissue engineering construct.

In some embodiments, assembly of an LBL film may involve a series of dip coating steps in which a substrate is dipped in alternating solutions. In some embodiments, LBL assembly of a film may involve mixing, washing or incubation steps to facilitate interactions of layers, in particular, for non-electrostatic interactions. Additionally or alternatively, it will be appreciated that LBL deposition may also be achieved by spray coating, dip coating, brush coating, roll coating, spin casting, or combinations of any of these techniques. In some embodiments, spray coating is performed under vacuum. In some embodiments, spray coating is performed under vacuum of about 10 psi, 20 psi, 50 psi, 100 psi, 200 psi or 500 psi. In some embodiments, spray coating is performed under vacuum in a range of any two values above.

Certain characteristics of a coated device may be modulated to achieve desired functionalities for different applications. Dose (e.g., loading capacity) may be modulated, for example, by changing the number of multilayer units that make up the film, the type of degradable polymers used, the type of polyelectrolytes used, and/or concentrations of solutions of agents used during construction of LBL films. Similarly, release kinetics (both rate of release and release timescale of an agent) may be modulated by changing any or a combination of the aforementioned factors.

In some embodiments, the total amount of agent released per square centimeter is about or greater than about 1 mg/cm². In some embodiments, the total amount of agent released per square centimeter in an LBL film is about or more than about 100 μg/cm². In some embodiments, the total amount of agent released per square centimeter in an LBL film is about or more than about 50 μg/cm². In some embodiments, the total amount of agent released per square centimeter in an LBL film is about or more than about 10 mg/cm², about 1 mg/cm², 500 μg/cm², about 200 μg/cm², about 100 μg/cm², about 50 μg/cm², about 40 μg/cm², about 30 μg/cm², about 20 μg/cm², about 10 μg/cm², about 5 μg/cm², or about 1 μg/cm². In some embodiments, the total amount of agent released per square centimeter in an LBL film is in a range of any two values above.

A release timescale (e.g., t_{50%}, t_{85%}, t_{99%}) of an agent for delivery can vary depending on applications. In some embodiments, a release timescale of an agent for delivery is less or more than about 1 hour, 2 hours, 3 hours, 4 hours, 5 hours, 10 hours, 15 hours, 20 hours, 25 hours, 30 hours, 40 hours, 50 hours, 75 hours, 100 hours, 150 hours, or 200 hours. In some embodiments, a release timescale of an agent for delivery is less or more than about 1 day, 2 days, about 5 days, about 10 days, about 12 days, about 20 days, about 30 days, 50 or about 100 days. In some embodiments, a release timescale of an agent for delivery is in a range of any two values above.

### EXAMPLES

### Example 1

### Release of Vancomycin from LBL Coated Substrates

In this Example, we have examined coating a commercial gelatin sponge with degradable polymer LBL films containing a model drug, vancomycin. The effect of LBL films on sponge absorption capabilities and the effect of the sponge on drug release kinetics were both examined. Application of vancomycin containing LBL assembled films to this highly porous substrate greatly increased drug loading up to approximately 880% compared to a flat substrate. Vancomycin drug release was extended out to 6 days compared to 2 days for film coated flat substrates. Additionally, the absorbent properties of the gelatin sponge were actually enhanced by up to 170% due to the presence of the vancomycin film coating. A comparison of film coated sponges with sponges soaked directly in vancomycin demonstrated the ability of the LBL films to control drug release. Film released vancomycin was also found to remain highly therapeutic with unchanged antimicrobial properties compared to the neat drug, demonstrated by quantifying vancomycin activity against Staphylococcus aureus in vitro.

Materials

Poly(β-amino ester) 2 was synthesized as previously described. Briefly, poly 2 was synthesized via reaction of 4,4- trimethylenedipiperidine with 1,6-hexanediol diacrylate in tetrahydrofuran at 50° C. for 48 hours. The polymer was subsequently precipitated in cold hexanes. The final poly 2 structure contains both amine groups providing the positive charge and ester bonds rendering the polymer degradable. Vancomycin and sodium acetate buffer (3 M) were purchased from Sigma-Aldrich (St. Louis, Mo.). Dextran sulfate sodium salt (Mₙ=500 kDa) was purchased from Polysciences (Warrington, Pa.). Dulbecco's phosphate buffered saline (PBS, 0.1 M) was purchased from Invitrogen (Carlsbad, Calif.). Deionized water (18.2 MΩ, Milli-Q Ultrapure Water System, Millipore) was utilized in all experiments. S. aureus 25923 was obtained from ATCC (Manassas, Va.). Cation-adjusted Mueller Hinton broth (CaMHB), Bacto agar, and vancomycin susceptibility test discs were obtained from BD Biosciences (San Jose, Calif.). Surgifoam® absorbent gelatin sponges (manufactured by Ferrosan and distributed by Johnson and Johnson) were generously donated by Ferrosan (Soeborg, Denmark).

Film Assembly on Gelatin Sponges

Vancomycin containing films were assembled using spray LbL assembly as previously described. Briefly, these films were constructed using a tetralayer architecture, denoted: (poly 2/dextran sulfate/vancomycin/dextran sulfate)ₙ, where n represents the number of tetralayers deposited (films with n=60 and 120 were assembled in this work). All deposition solutions were formulated at a concentration of 2 mg/mL in 0.1 M sodium acetate buffer (pH 5). Films were assembled using a programmable spraying apparatus (Svaya Nanotechnologies). The gelatin sponge was used as received with no pretreatment. A 50 psi vacuum was applied to the back of sponge (with dimensions of 1 cm×5.5 cm×4.5 cm) during the LbL deposition process. For each tetralayer, each deposition step lasted 2 seconds, followed by a 3 second rinse with 0.1 M sodium acetate buffer (pH 5) at a flow rate of 0.25 mL/s. Following film deposition, the gelatin sponge was allowed to dry using gentle house vacuum and then stored at 4° C. prior to subsequent analysis. For contact angle and swelling measurements, films were coated on silicon substrates without vacuum application.

Characterization of Film and Gelatin Sponge Properties

Advancing water contact angle of 60 and 120 tetralayer films coated on silicon wafers was obtained using a standard sessile drop technique with a VCA 2000 video contact angle system and the accompanying VCA OptimaXE software (AST Products, Inc.). Additionally, film swelling was monitored for the 60 tetralayer film using a MultiMode 8 scanning probe microscope with a Nanoscope V controller (Veeco Metrology) operated in tapping mode. Dry film thickness measurement was made by scanning over a region containing both the film and a deliberate scratch. Change in film thickness was monitored upon introducing 0.01 M PBS into a liquid chamber and waiting for approximately 60 seconds prior to starting the measurement.

Gelatin sponge morphology before and after LbL spray coating was examined using scanning electron microscopy (JEOL JSM-6060). The surface area of uncoated sponges was evaluated using an accelerated surface area and porosimetry analyzer (Micromeritics ASAP 2020). This measurement utilizes the gas sorption method, in which first the sponge is cleaned via degassing and then filled with a gas until the entire pore volume of the sample has been filled. The BET method is then applied to estimate surface area from data on the mass of gas adsorbed. Additionally, the ability of the gelatin sponges to absorb 0.01 M PBS before and after LbL coating was examined. Pieces of coated and uncoated sponges were weighed and subsequently submerged in 10 mL of 0.01 M PBS for 10 minutes. Following this, the sponge was removed from the solution and weighed again. The difference is mass before and after soaking corresponded to the mass of PBS absorbed by the sponge. This value was normalized by the initial mass of the sponge, to give a measure of absorbency in milligrams of PBS per milligram of sponge. An absorbency ratio of LbL coated to uncoated gelatin sponges was calculated using the following equation:

$$Absorbency ratio = \frac{{( mg PBS absorbed / mg sponge )}_{LbL coated}}{{( mg PBS absorbed / mg sponge )}_{uncoated}}$$

Vancomycin Release from Gelatin Sponges

After LbL spray deposition, the coated gelatin sponge was cut into smaller pieces using a razor blade (with dimensions of approximately 0.7 cm×0.8 cm×1 cm). Each piece of sponge was released in 1 mL of 0.01 M PBS at 37° C. At predetermined times, the PBS was removed and frozen at −20° C. before subsequent analysis; a fresh 1 mL of PBS was added to continue film release. These film release solutions were monitored with high performance liquid chromatography (Agilent Technologies HPLC, 1100 series) using a C18 reverse phase column (Supelco) coupled with fluorescence detection, as previously described. Briefly, each sample was run for 10 minutes using a 70/30 0.01 M PBS/methanol mobile phase, 1 mL/min flow rate, 500 μL injection volume, and an excitation wavelength of 280 nm and emission wavelength of 355 nm for vancomycin detection. Fluorescence peak height was correlated with standards of known vancomycin concentrations and used to determine drug concentrations in coated sponge release samples. A piece of uncoated sponge was also released similarly to the coated sponges and examined with the same HPLC protocol, to ensure that potential peaks from sponge degradation did not interfere with vancomycin peaks (no interference was noted).

Release of vancomycin from gelatin sponge pieces containing drug but no LbL coating was also examined. After determining the total vancomycin loading in the 60 and 120 tetralayer LbL film coated sponges, these quantities of vancomycin were dissolved in deionized water and allowed to soak completely into pieces of non-film coated sponge. Immediately after soaking, these sponge pieces were released in PBS in the same way that LbL film-coated sponges were released; release was quantified using HPLC as described above.

Bacterial Growth Inhibition

The ability of LbL coated gelatin sponges to inhibit the growth of S. aureus 25293 was examined by exploring the activity of the LbL coated sponge directly as well as drug release solutions using previously described methods. Briefly, coated sponge activity was assessed directly using a modified Kirby-Bauer test on a bacteria coated agar plate. Here, pieces of coated sponge, along with controls of uncoated sponge and vancomycin susceptibility discs (30 μg), were applied to CaMHB-agar plates which were evenly coated with S. aureus in its exponential growth phase at a concentration of 10⁸ CFU/mL. These plates were incubated at 37° C. for 16-18 hours and observed for zones of inhibition surrounding the test samples following incubation.

A quantitative determination of vancomycin activity from a coated gelatin sponge sample was obtained by first completely releasing the coated sponge into a 1 mL, 0.01 M PBS bath, at 37° C. The exact concentration of vancomycin in this release solution was determined using HPLC methods described earlier. Subsequently, S. aureus in its exponential growth phase was added to dilutions of this release solution in CaMHB at a final concentration of 10⁵ CFU/mL. Non-film released vancomycin was also tested. Additionally, controls of 0.01 M PBS dilutions in media containing no drug exposed to S. aureus (positive control) and not exposed to S. aureus (negative control) were included. These dilutions were incubated at 37° C. for 16-18 hours with agitation, following which, optical density at 600 nm was read using a BioTek PowerWave XS plate reader. Normalized bacteria density was calculated as follows:

$$Normalized S . aureus density = \frac{( {OD}_{600 sample} - {OD}_{600 negarive control } )}{( {OD}_{600 positive control} - {OD}_{600 negative control} )}$$

Statistical Analysis

All experiments performed in this work were done in triplicate at minimum. Data is reported as mean±standard deviation. Gelatin sponge morphology via SEM was examined for a minimum of three separate samples per test condition at a minimum of three locations per sample.

Gelatin Sponge Coating Characterization

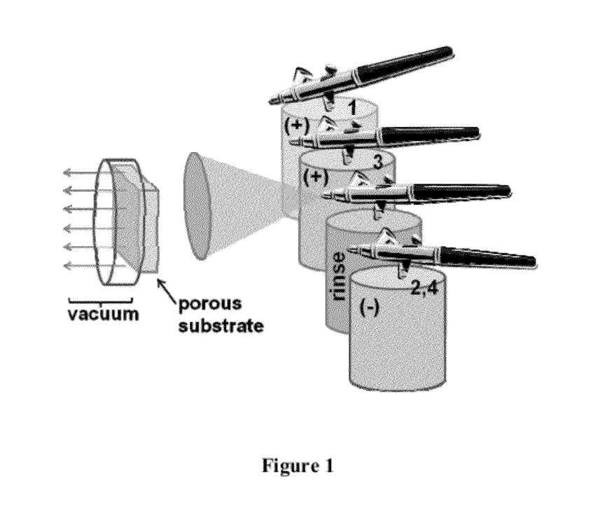

In the LbL self-assembly process, substrates are coated with materials that have complementary functionality (i.e. charge, hydrogen bonding interactions, etc.) one layer at a time. Rinsing between deposition steps removes non-specifically bound material. We used this technique to coat commercial gelatin sponges with antibiotic releasing LbL films. Type A gelatin which is processed to create the gelatin sponges used in this work is positively charged below its isoelectric point of approximately 8. If these characteristics of the gelatin are maintained in the gelatin sponge, one would expect the sponge to interact strongly with the first negatively charged component deposited upon it. Here, that component was dextran sulfate, a highly negatively charged biopolymer. Due to the fact that these commercial gelatin sponges are highly water absorbent, the sponges were coated with spray LbL assembly rather than the traditional dip assembly technique. In spray LbL assembly, each film component and rinse solution is aerosolized and propelled at the substrate being coated as shown in FIG. 1. Due to the fact that the process is not diffusion limited, spray LbL significantly reduces the overall assembly time compared to the dip LbL technique, from approximately 40 minutes per tetralayer to 20 seconds per tetralayer. As shown in FIG. 1, a vacuum was applied to the back of the porous gelatin substrate to take advantage of the large surface area available for coating while sweeping liquid through the substrate at a fast rate during the spray process. For the 60 and 120 tetralayer films deposited in this work, it takes 20 and 40 minutes to complete film spray assembly compared to 40 and 80 hours for dipping. Sponges were soaked in the buffer solution used in film assembly for the same 20 and 40 minute time period. There was no significant change in sponge dimensions and 8.2±1.4 and 11.5±1.5 milligrams of buffer were absorbed per milligram of sponge during the 20 and 40 minute soak period, respectively. Spray assembly does not require complete immersion of the sponge in liquid for lengthy times, which avoids this significant absorption that may lead to inadequate coating and contamination from carryover of deposition solutions. Vacuum application further eliminates these complications. Overall, the short and significantly drier spray process, allows for effective coating of the porous and absorbent gelatin sponges.

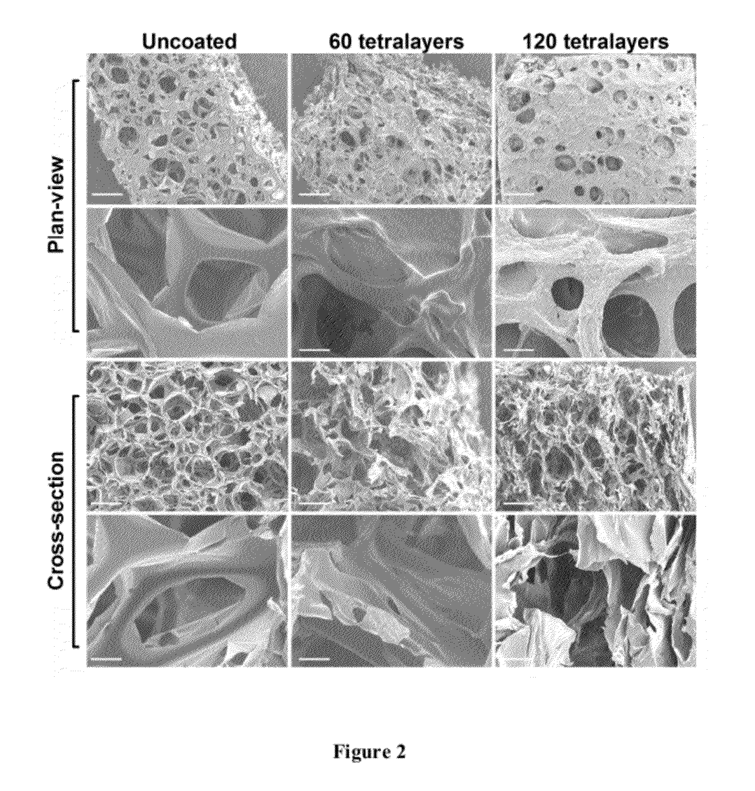

Prior to and after spray coating with antibiotic LbL films, scanning electron microscopy was used to examine dry sponge morphology. FIG. 2 shows both plan-view and cross-sectional SEM micrographs of uncoated sponges and sponges coated with both 60 and 120 tetralayers of (poly 2/dextran sulfate/vancomycin/dextran sulfate)ₙ films. Poly 2 is a cationic and hydrolytically degradable poly(β-amino ester), vancomycin is the cationic antibiotic, dextran sulfate is a counter polyanion, and n is the number of tetralayers deposited. The properties of these films on flat, non-porous, and non-absorbent substrates have previously been described. FIG. 2 shows the highly porous nature of the gelatin sponge. The uncoated sponge surface area was found to be approximately 5.3 m²/g; this area was taken as the maximum surface area available for film coating and drug release. As evidenced in FIG. 2, the underlying sponge morphology is maintained following the spray LbL process; there is no evidence of collapse, expansion, or degradation of the sponge microstructure. The only visible difference between the coated and uncoated samples is the presence of the coating itself, which, as expected, appears thicker for the 120 tetralayer films compared to the 60 tetralayer films. As the coating grows, it lines the pore surfaces and eventually begins to partially fill gaps between pores.

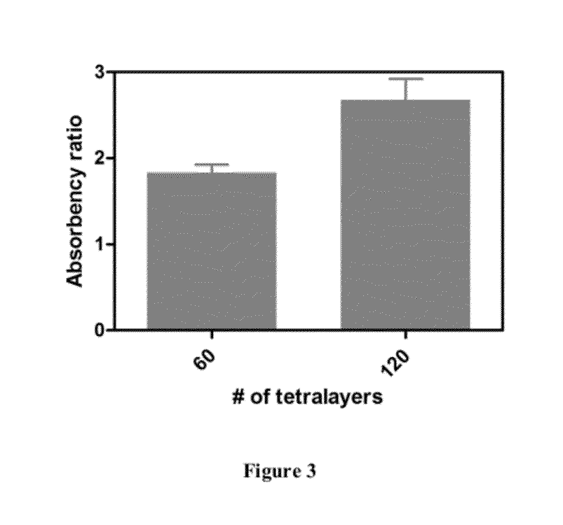

To ensure that the coated sponge maintains its primary function, the absorption of aqueous phosphate buffered saline (PBS) by LbL coated versus uncoated sponges was examined. The ratio of mass of PBS absorbed by LbL coated versus uncoated sponges is shown in FIG. 3. The LbL coating greatly enhances the absorption of PBS per milligram of sponge. The 60 tetralayer LbL coating increased liquid absorption by approximately 80% (from 10.9 mg PBS/mg uncoated sponge to 19.7 mg PBS/mg coated sponge), while the 120 tetralayer coating increased sponge absorption by 170% compared to uncoated sponges (from 6.7 mg PBS/mg uncoated sponge to 18.0 mg PBS/mg coated sponge). Note that variations in uncoated sponge PBS absorption were frequently observed; therefore, prior to coating, a piece of the gelatin sponge was extracted from the sponge to be coated, and its PBS absorption capability was determined which was subsequently compared to absorption of the sponge after coating. It is generally known that polyelectrolyte multilayers can swell and take up significant amounts of water when hydrated. In fact, the 60 tetralayer vancomycin containing LbL films instantaneously swell to approximately 180% of their dry film thickness in PBS when assembled on a flat substrate. Additionally, the advancing water contact angle on flat substrates coated with 60 and 120 tetralayer vancomycin LbL films, was measured to be 109.6±6.2° and 53.1±9.2°, respectively, further demonstrating the hydrophilicity of these films, especially at 120 tetralayers. The difference in contact angle observed between the 60 and 120 tetralayer films is likely due to increasing interdiffusion that occurs within species in these films at higher tetralayer numbers, leading to a change in film surface properties. Gelatin hydrogels have been reported to have high advancing contact angles of approximately 130°, depending on gelatin concentration, despite their large water absorption capabilities. It is clear from this data that the LbL film within the coated sponge is able to enhance its water uptake, likely through increased wettability and capillarity of the sponge pores and the increased thickness of the coating itself, which can add to the total amount of water absorptive material. Overall, the LbL coating lacks any detrimental effect on the functions of the commercial gelatin sponge and actually enhances the sponge absorbency by a factor greater than approximately 2.

Vancomycin Release and Therapeutic Potential

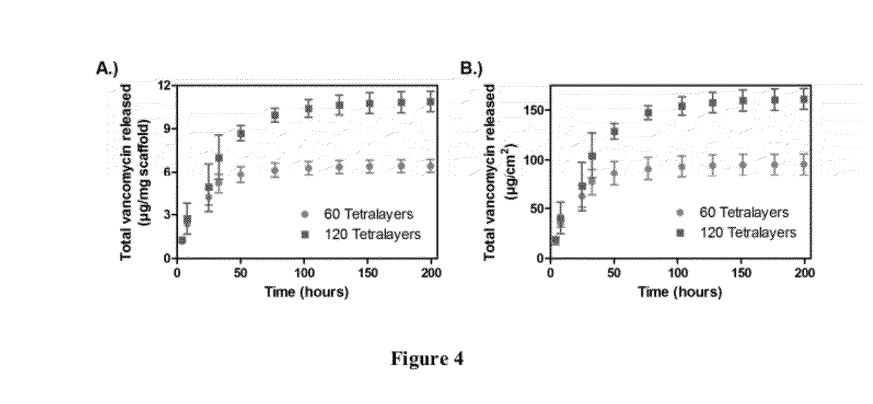

The effects of the micro and nanoscale structure of drug releasing devices on dictating drug release kinetics have been thoroughly explored. The ability to generate uniform conformal coatings within complex porous scaffolds allows us to use the pore morphology of the underlying substrate as a means of modulating the release behavior of our LbL films; here we have explored the impact of the gelatin sponge morphology on vancomycin loading and release kinetics. FIG. 4 shows the release profiles of vancomycin from 60 and 120 tetralayer spray LbL coated sponges at physiologic conditions (PBS, pH 7.4, 37° C.). Two different representations of the data are shown; FIG. 4A shows total vancomycin released per milligram of the sponge, while FIG. 4B shows the total vancomycin released per square centimeter of projected in-plane sponge surface. Both of these methods of data representation provide valuable information about the final drug loading capabilities of these LbL films. Compared to flat substrates which were previously sprayed with the same vancomycin containing LbL film and found to contain 9.7±1.0 μg/cm² and 20.0±1.9 μg/cm² for n=60 and 120, respectively, films sprayed on gelatin sponges showed an 880±140% and 710±85% greater vancomycin loading for n=60 and 120, respectively. This significant increase in drug loading comes from the increased overall surface area of the gelatin sponge. From FIG. 2 it is clear that there is significant bridging of the sponge pores by the multilayer film coating. The LbL film is observed to penetrate across the 1.0 cm thickness of the sponge, but with less deposition in regions furthest from the front surface during the spray LbL process. It is conceivable that if complete conformal coverage of all available surface area were achieved, the loadings and release times could be increased even further.

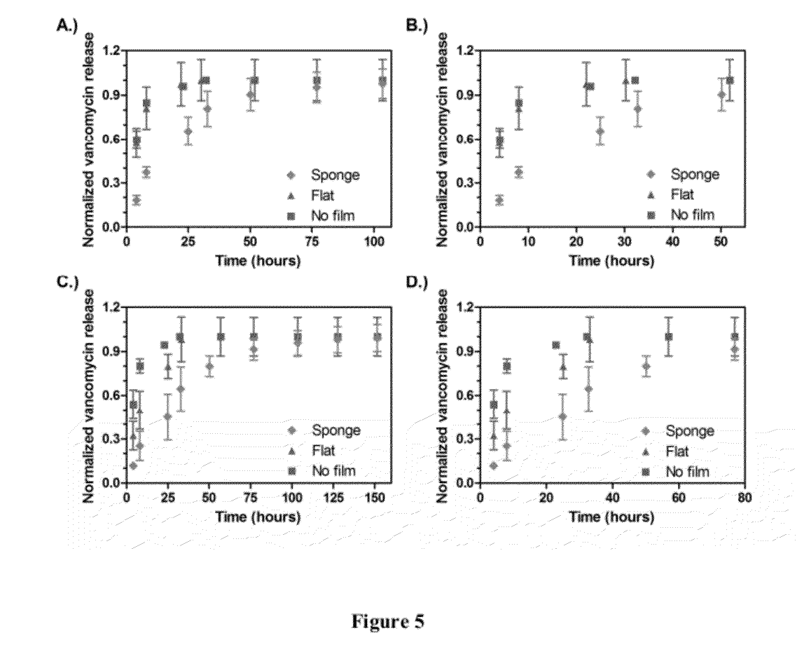

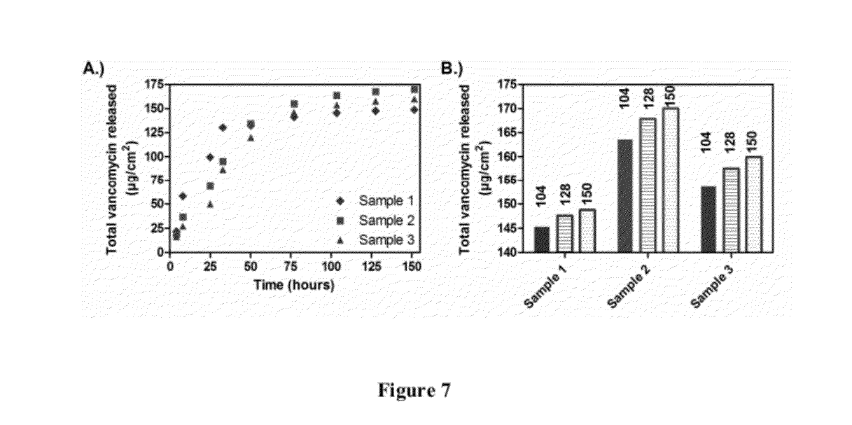

FIG. 5 shows normalized release profiles for both gelatin sponges and flat substrates coated with (poly 2/dextran sulfate/vancomycin/dextran sulfate)ₙ where n=60 and 120. The release at each time point is normalized to the final vancomycin loading in the film. FIGS. 5A and 5B show full and partial release data for 60 tetralayer films, while FIGS. 5C and 5D show the full and partial release data for 120 tetralayer films. These figures also show release profiles from gelatin sponges that were soaked with vancomycin and released immediately into PBS (no LbL film). This condition was used to simulate an option that might be commonly employed by a surgeon, reconstituting a lyophilized formulation of vancomycin in solution and soaking up the solution with the gelatin sponge immediately prior to using the sponge to absorb blood during an invasive procedure. Table 1 below summarizes several relevant release timescales that can be obtained from the graphs in FIG. 5, namely, the time for 50, 85, and 99% of the drug to be released (t_{50%}, t_{85%}, t_{99%}) from LbL coated flat substrates and gelatin sponges along with non-LbL coated sponges. The relevant release timescales were determined by examining each sample that contributed to the averages and standard deviations plotted in FIG. 5, separately. FIG. 7 shows an example of three separate 120 tetralayer LbL film coated sponge samples that were released (FIGS. 5C and 5D show the averaged release and standard deviations of these three samples). From FIGS. 7A and 7B we can see that although the averages of these individual samples may overlap for consecutive time points (especially towards later time points), the vancomycin quantity in each individual release sample increases significantly. Once significant increase in vancomycin quantities between one or more samples for consecutive time points was not visible, release was considered to be complete.

TABLE 1
Vancomycin release kinetics.

| | Substrate | t_{50%} (hours) | t_{85%} (hours) | t_{99%} (hours) |
|---|---|---|---|---|
| No film^{a} | Gelatin sponge | <4 | 8 | 24 |
| n = 60^{b} | Gelatin sponge | 16 | 40 | 104 |
| | Flat | <4 | 10 | 24 |
| n = 120^{b} | Gelatin sponge | 28 | 63 | 150 |
| | Flat | 8 | 27 | 45 |

^{a}Sponge soaked in vancomycin (no LbL coating).
^{b}Film architecture: (poly 2/dextran sulfate/vancomycin/dextran sulfate)ₙ.

Together FIG. 5 and Table 1 show that similar to drug loading, there are significant differences in vancomycin release kinetics for both the 60 and 120 tetralayer LbL film coatings on gelatin sponges compared to flat substrates. For both the 60 and 120 tetralayer film coated sponges, there is an approximate 4-fold increase in t_{50%}. Final sponge LbL drug release lasts over approximately 104 and 150 hours versus 24 and 45 hours for the same 60 and 120 tetralayer films, respectively, on flat substrates. On flat substrates, the 120 tetralayer film was previously shown to have a period of linear release lasting from 4 to 33 hours, compared to a rapid bolus release of vancomycin from a 60 tetralayer film. These differences were attributed to an increased level of interdiffusion between film components at 120 tetralayers compared to 60 tetralayers, which appeared to promote non-electrostatic secondary interactions between vancomycin and dextran sulfate, stabilizing these films and increasing the timescale for release. Similar to what was seen on flat substrates, the 120 tetralayer sponge coatings have a much more linear release profile than the 60 tetralayer sponge films and release vancomycin over a longer period of time; the linear release period lasts from 4 to approximately 60 hours (R²=0.97) at which point approximately 90% of the vancomycin has been released from the films. Following this linear release period, there is a gradual decline in drug release until it reaches zero at approximately 150 hours. Similar to release from film coated flat substrates, sponge film coatings are expected to release drug based both on the hydrolytic degradation of poly 2 within the film architecture along with drug diffusion from the film. The increased linearity of release for the 120 tetralayer film coated sponges is likely due to increase in film component interdiffusion compared to the 60 tetralayer film, which further stabilizes these films similar to what was seen on flat substrates. Overall, the increased tortuosity of the porous gelatin sponges appears to greatly increase the vancomycin release timescales from these previously developed LbL spray coatings from 1 to 2 days on non-porous substrates to 4 to 6 days on this clinically relevant substrate.

Additionally, FIG. 5 and Table 1 show comparisons between drug release from sponges soaked in vancomycin compared to those in which the LbL film was used to encapsulate drug and coat the sponge. Both the 60 and 120 tetralayer films show a significant improvement in controlling drug release from sponges compared to simply soaking the sponge in vancomycin and releasing. For sponges soaked in drug, 60% of the loaded vancomycin is released in 4 hours and all of the drug is completely released at 24 hours. The t_{50%} value is 4 and 7-fold greater for the 60 and 120 tetralayer LbL sponge coatings, respectively, compared to the soaked sponge. This provides strong support for the use of these LbL films for controlling drug release from coated substrates.

The release profiles and drug loadings of 60 and 120 tetralayer LbL film coated gelatin sponges shown in this work have the potential to be highly therapeutic. Two of the primary causes for development of antibiotic resistance and difficulty in treating infection are drug concentrations below the minimum inhibitory concentration (MIC) of the drug against a particular pathogen and inappropriate delivery timescales. With the sponge coatings developed here, we obtain tunable drug release over multiple days which can be suitable for both eradicating (rapid drug release required over approximately 24 hours) and preventing infection from occurring at a wound site (drug delivery needed over a minimum of several days). The timescales of vancomycin delivery that have been demonstrated in this work are comparable to current commercially available antimicrobial dressings that have been used for the effective treatment of various wounds, including burns; these dressings typically deliver drugs over approximately 3 days. Additionally, the large drug loadings of the sponge coatings developed here can lead to local vancomycin concentrations well above the MIC of vancomycin against common bacteria, including S. aureus.

Drug Activity

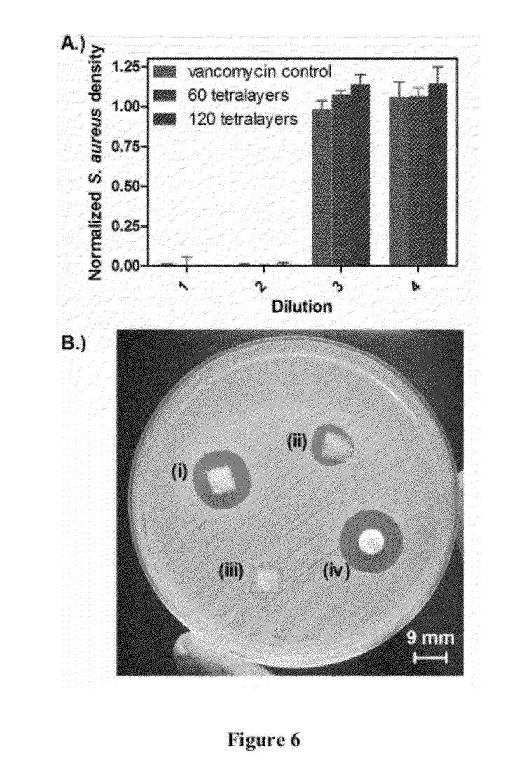

Upon LbL coating of the gelatin sponges and quantifying the release kinetics of vancomycin from these sponges, we established the therapeutic activity of these coatings in vitro. First, we released samples of 60 and 120 tetralayer coated sponges in PBS and examined the efficacy of those release solutions in inhibiting S. aureus growth. FIG. 6A shows the normalized density of S. aureus exposed to dilutions of vancomycin released from these coatings, along with a standard control solution of vancomycin that was not released from a film coating. Vancomycin released from coated gelatin sponges completely maintains its activity against S. aureus, with an MIC between 0.5 to 2 μg/mL as expected for non-film released vancomycin.

Activity was also assessed directly upon exposing pieces of coated gelatin sponges to S. aureus coated agar for a modified Kirby-Bauer test. FIG. 6B shows the results of testing a 60 tetralayer film coated sponge (i and ii) along with an uncoated control (iii) and a vancomycin control disc (30 μg, iv). Sample (i) and (ii) come from the same piece of film coated sponge, which was 1 cm thick. Upon coating, the sponge was sliced into two pieces, such that sample (i) represents the 0.67 cm thick slice of sponge containing the face that was directly exposed to the aerosolized spray from the LbL apparatus. Sample (ii) is the remaining foam from underneath this portion (0.33 cm thick). A clear zone of inhibition (ZOI) surrounds the vancomycin control (iv) along with both coated sponge pieces (i) and (ii), visually showing the inhibition of S. aureus growth by these samples. The presence of a ZOI surrounding sample (ii) confirms that vacuum application during LbL deposition allows penetration of the film components throughout the thickness of the gelatin sponge; however, the surface coverage and film thickness of the scaffold is highest on the front face of the membrane, and lower on the back face. The homogeneity of the coating may be improved in the future by application of a higher pressure vacuum during film deposition or by spray LbL coating both sides of the sponge. As expected, there is no ZOI surrounding the uncoated gelatin sponge. Based on the results of these in vitro assays, it is clear that the vancomycin LbL coating of commercial gelatin sponges renders the sponge highly antimicrobial and effective against a common source of infection, S. aureus.

Here we have demonstrated the application of a vancomycin releasing multilayer film to a clinically relevant and commercially available, highly absorbent and porous gelatin sponge. Here, we have used spray LbL assembly to coat gelatin sponges and show that the substrate has a tremendous impact on increasing vancomycin loading and also extending release times compared to a flat substrate. Additionally, we have shown that the LbL films significantly increase the ability of the sponge to absorb liquid. This work has demonstrated that spray LbL assembly is a versatile tool that can be applied to a variety of substrates, even in the case of water-absorbable biomaterials. Using this technique, we have enhanced the therapeutic properties of a commercial gelatin sponge, rendering it antimicrobial while increasing its absorption capabilities. In a similar fashion, multiple substrates such as sutures, bandages, and nanofiber matrices can be coated with therapeutics rapidly and effectively to generate sustained drug release biomedical coatings in many embodiments of the present disclosure.

### Example 2

### (Layer-By-Layer) LBL Coated Substrates for Hemostasis

Characteristics of layer-by-layer (LbL) films (such as, film stability, release kinetics of agents, etc.) vary depending on mechanisms and materials used to construct the films. In this work, spray LBL assembly is used to create exemplary hemostatic films with alternating layers of thrombin and tannic acid. Use of spray assembly technique enables coating of porous and absorbent commercial gelatin sponges with LBL films. Coated sponges are able to promote instantaneous hemostasis in a porcine spleen bleeding model.

Materials: Branched polyethyleneimine (BPEI, Mₙ=50-100 kDa) was obtained from Polysciences (Warrington, Pa.). Tannic acid and mannitol were obtained from Sigma-Aldrich (St. Louis, Mo.). Dulbecco's phosphate buffered saline (PBS, 0.1 M) was purchased from Invitrogen (Carlsbad, Calif.). TCNB buffer (pH 7.5) was formulated in deionized water containing 50 mM Trizma, 1.1 mM calcium chloride, 150 mM sodium chloride, 0.05% Brij® 35, and 0.2 g/L bovine serum albumin each obtained from Sigma-Aldrich (St. Louis, Mo.). Silicon substrates (test grade, n type) were obtained from Silicon Quest International (Santa Clara, Calif.). Quartz crystal microbalance (QCM) sensors (silicon dioxide coated, 50 nm) were purchased from Q-Sense (Biolin Scientific, Linthicum, Md.). High purity bovine thrombin powder (12.6% protein, 87.4% mannitol and sodium chloride, BioPharm Laboratories, Bluffdale, Utah) and Surgifoam® absorbent gelatin sponges were generously donated by Ferrosan Medical Devices A/S (Soeborg, Denmark). Deionized water (18.2 MΩ, Milli-Q Ultrapure Water System, Millipore) was utilized in all experiments.

Film Preparation: Films were prepared using spray LbL assembly with a programmable spray apparatus (Svaya Nanotechnologies) as previously described. The film architecture was denoted (thrombin/tannic acid)ₙ, where n represents the number of bilayers deposited. Films were assembled on silicon in order to characterize film growth, morphology, and dissolution characteristics and on gelatin sponges in order to examine efficacy. Prior to assembly on silicon, substrates were cleaned with deionized water, methanol, and water again, and dried under nitrogen. The substrates were then plasma etched with air in a Harrick PDC-32G plasma cleaner at high RF level for 60 seconds. Immediately following plasma etching, the substrates were submerged in BPEI solution (2 mg/mL, pH 7.4, in 0.01 M PBS) for 20 minutes. Following this, substrates were washed with 0.01 M PBS (pH 7.4) and dried under nitrogen. The bilayer film was then deposited, by spraying thrombin (1 mg/mL, pH 7.4, in 0.01 M PBS) followed by tannic acid (2 mg/mL, pH 7.4, in 0.01 M PBS) each for 20 seconds at a flow rate of 0.25 mL/s. Following each deposition step, a 5 second wash with 0.01 M PBS (pH 7.4) was sprayed at a flow rate of 0.25 mL/s. For depositing films on gelatin sponges, a 50 psi vacuum was applied to the back of the sponge during the spray process. The sponge (approximately 1 cm×5.5 cm×4.5 cm) was first sprayed with BPEI for 20 seconds, followed by a 5 second PBS rinse. The bilayer film was then deposited on the sponge with the same solution concentrations and spray timings as with the flat substrates. Films assembled on silicon were dried under nitrogen, while film coated sponges were allowed to dry completely on gentle house vacuum. All films were stored dry at 4° C. prior to subsequent analysis.

Characterization of Film Properties: Initially, film growth was characterized by using quartz crystal microbalance with dissipation monitoring (QCM-D, Q-Sense E4). Silicon dioxide coated sensors (with fundamental frequency of 4.95 MHz±50 kHz) were rinsed with deionized water, methanol, and water again, and dried under nitrogen prior to use. Prior to deposition, the sensors were UV-ozone treated for 20 minutes using a UV-Ozone ProCleaner (Bioforce). The sensor was placed in a flow cell and changes in frequency and dissipation were monitored while flowing in film deposition solutions at 150 μL/min. The same solution concentrations and order of deposition steps was used in QCM-D film growth studies as with spray film deposition. Total flow time was 5 minutes for the initial BPEI deposition, 15 minutes for thrombin, and 10 minutes for tannic acid, with 5 minute PBS rinse steps between each deposition; a 5 bilayer film was deposited. To determine if mannitol contributed to film growth, assembly of a control film with architecture (mannitol/tannic acid)₅, was attempted. The mannitol (1 mg/mL, pH 7.4, in 0.01 M PBS) deposition step was 15 minutes long.

Spray film growth was monitored via profilometer (Dektak 150 Stylus Profiler, Bruker AXS). Following spray film deposition on silicon substrates at varying bilayer numbers, films were scored with a razor and tracked over a 700 μm scan length to measure film thickness. The surface morphology of these films was monitored using a Dimension 3100 atomic force microscope with Nanoscope 5 controller (Veeco Metrology) operated in tapping mode over 10 μm by 10 μm areas. Root mean squared (RMS) roughness values were obtained using Nanoscope Analysis 1.10 software (Veeco). Morphology of films sprayed on gelatin sponges were examined with scanning electron microscopy (JEOL JSM-6060).

Dissolution of films assembled on silicon substrates with n=10, 25, and 50, was also monitored. These films (approximately 1 cm²) were soaked in 500 μL of 0.01 M PBS at 37° C. At predetermined times, the films were removed from solution, dried under nitrogen, and film thickness was determined using a profilometer as described earlier. This study was carried out over 240 hours.

The absorption of 0.01 M PBS by gelatin sponges was characterized before and after film coating for n=10, 25, and 50. Sponges were weighed and then submerged in 10 mL of 0.01 M PBS for 10 minutes. Subsequently the sponge was removed from the solution and weighed again. The difference in mass before and after soaking corresponded to the mass of PBS absorbed by the sponge.

Film Activity: Film activity was assessed both in vitro and in vivo for sponges coated with bilayer films (n=10, 25, and 50). First, coated sponges were soaked in 10 mL of TCNB buffer at room temperature under agitation for times varying from 10 minutes to 6 days. Following this soak procedure, the release solutions, along with standards of thrombin, were tested using an automated coagulation analyzer (START 4, Diagnostica Stago) in which the time for clot formation is measured once fibrinogen (10 mg/mL) solution is added to a sample, by monitoring the movement of a metal ball in solution between an applied magnetic field.

In vivo activity of film coated sponges (n=10, 25, and 50) was determined in a porcine spleen bleeding model. Controls of untreated gauze and sponges coated with a single monolayer of BPEI, were also tested. All animal tests were performed in accordance with protocols approved by the Committee on Animal Care (Massachusetts Institute of Technology). Danish country breed pigs were used in these studies. Prior to surgery, pigs (approximately 40 kg) were sedated and provided preoperative analgesia via intramuscular injection of a Zoletil® mixture (0.1 mL/kg). This mixture was formulated from Zoletil 50® (125 mg Tiletamine and 125 mg Zolazepam) dissolved in 2.5 mL Turbogesic® (Butorphanol, 10 mg/mL), 1.25 mL Ketaminol® (Ketamin, 100 mg/mL), and 6.25 mL Rompun® (xylazinhydrochloride, 20 mg/mL). Intraoperative anesthesia was maintained by intravenous administration of Propofol (10 mg/mL, 1 mL/kg/hour) and Fentanyl (0.05 mg/mL, 0.5 mL/kg/hour). The anesthesia and analgesia regimen used here is known not to affect hemostasis. Following anesthesia, pigs were intubated and ventilated with a mixture of 0.5 L oxygen/2.5 L air/min. Pigs were kept fully hydrated with infusion of lactated Ringer's solution (125 mL/hr).

Following anesthesia, the porcine spleen injury model was prepared. A midline abdominal incision was made to expose the spleen. The spleen injury was induced with a punch incision (8 mm wide and 3 mm deep). The bleeding intensity was evaluated on a 0 to 5 scale, where: level 0 indicates no bleeding (for at least 30 seconds), level 1 indicates no bleeding initially followed by bleeding (within the first 30 seconds post injury), level 2 indicates bleeding (site filling in approximately 30 seconds), level 3 indicates bleeding (site filling in approximately 3 seconds), level 4 indicates bleeding (site filling immediately with no arterial or pulsating bleeding), and level 5 indicates bleeding (site filling immediately with arterial or pulsating bleeding). Only wounds classified as level 4 or 5 were utilized in this study. A new incision was created for each test sample (up to 16 samples were evaluated per pig). Immediately following bleeding intensity evaluation, the test sample (2 cm×2 cm piece of film coated sponge, BPEI coated sponge, or untreated gauze, wet with 0.8 mL of 0.9% saline solution) was placed directly on the injury and even digital pressure was applied for 60 seconds. The site was monitored for up to 120 seconds. If bleeding was not observed in this time following compression, hemostasis was achieved. However, if bleeding occurred within the 120 seconds following compression, digital compression was applied again for 30 seconds, and the injury was monitored. Digital compression and observation were repeated until hemostasis was achieved (classified as 120 seconds free of bleeding) or until the test period reached 12 minutes (classified as an ineffective sample). The final result of time to hemostasis was defined as the total testing time to achieve hemostasis minus the final hemostasis evaluation period. Pigs were euthanized with intravenous pentobarbital (300 mg/mL, 0.1 mL/kg) at the completion of the study.

Statistical Analysis: Film properties on silicon substrates and sponge coating morphology and absorption capabilities were evaluated for a minimum of three samples per each bilayer number. In vitro activity testing was conducted for a minimum of three samples per bilayer number. In vivo activity was monitored for 9 samples per each bilayer number and 9 controls. All data presented here is represented as mean±standard deviation of these multiple trials. Data fitting and analysis were conducted using GraphPad Prism 5 software.

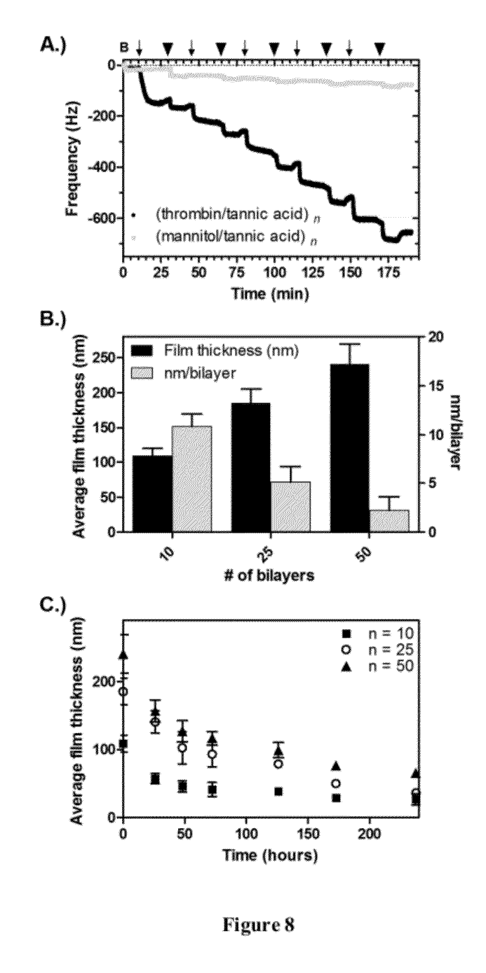

Sprayed (Thrombin/tannic acid)ₙ Film Dissolution: Thickness data shown in FIG. 8C for sprayed (thrombin/tannic acid)ₙ films exposed to 0.01 M PBS at 37° C. over approximately 10 days was fit with the following equation for one-phase decay:

Y=Yₒq^{-kt}

Here, Y is the film thickness, Yₒ is the initial film thickness, t is time, and k is the rate constant (hours⁻¹). Using this model we estimated the dissolution rate constants for these films in PBS and the dissolution half-life (t_{1/2}). These parameters, along with the average film thickness, RMS roughness, and the coefficient of determination (R²) for the fit, are shown in Table 2 for n=10, 25, and 50,

TABLE 2
Sprayed (thrombin/tannic acid)ₙ film characteristics.

| | n = 10 | n = 25 | n = 50 |
|---|---|---|---|
| Average film thickness (nm) | 108.6 ± 12.4 | 185.6 ± 19.6 | 240.9 ± 28.4 |
| RMS roughness (nm) | 46.3 ± 3.7 | 51.9 ± 4.2 | 66.8 ± 11.5 |
| Yₒ (nm) | 89.0 | 173.3 | 208.1 |
| k (hours⁻¹) | 8.2 × 10⁻³ | 7.5 × 10⁻³ | 6.5 × 10⁻³ |
| t_{1/2} (hours) | 84.8 | 92.6 | 107.3 |
| R² | 0.75 | 0.95 | 0.87 |

To develop this hemostatic coating we have applied the layer-by-layer (LbL) assembly technique in which sequential adsorption of materials with complementary functionality yields a multilayer film. Unlike traditional bulk polymer systems which are limited in their therapeutic loading capacity and often utilize processing conditions that are unsuitable for protein loading, LbL assembly has shown great versatility in encapsulation and delivery of biologically active materials. Additionally, LbL assembly and especially the newer spray LbL assembly technique, which was utilized in this work, can be used to directly coat the nano and microscale features of existing scaffold materials, enhancing the functionality of these substrates. This is especially applicable for rapid hemostasis, where optimized absorbent bandages have been commercially available for several decades and would benefit tremendously from pre-functionalization with hemostats. In this work, we report the first application of LbL assembly towards formulating films that instantaneously promote hemostasis and that can be applied to existing biomedical scaffolds. We discovered that we could use hydrogen bonding interactions between an essential clotting factor, thrombin (factor IIa), and a small polyphenol that is a component in black tea, tannic acid, to build films containing large thrombin loadings. Although electrostatic interactions have been used in the past to assemble LbL films containing a protein and a small molecule, here we demostrate that multilayer coatings can be assembled based on hydrogen-bonding interactions without incorporation of any synthetic polymers, further maximizing the thrombin density in these films. Additionally, both tannic acid and the bovine thrombin used in development of these films, are approved by the FDA, facilitating eventual clinical translation. To demonstrate the versatility and practical applicability of these LbL films, we applied them to a clinically available porous and absorbent gelatin sponge, which is typically soaked in thrombin immediately prior to use. We show that these LbL film coated sponges promote rapid hemostasis in a porcine spleen injury model while preserving the sponge absorption capabilities.

Thrombin is a large protein with an isoelectric point between pH 7.0 and 7.6. At conditions deviating from physiologic pH of 7.4, thrombin has been shown to degrade. As electrostatic interactions cannot be used to incorporate thrombin into an LbL film at conditions where the protein is stable, we explored hydrogen bonding as an alternative means of multilayer assembly, which has also been demonstrated to be useful in the construction of LbL films for biomedically relevant applications. Tannic acid is a polyphenol found in a variety of food products and stains and known to have antitumor, antibacterial, and antioxidant activity, as well as reported interactions with proteins. It has an abundance of hydrogen bond donating phenols and a reported pKa near 8.5. In accordance with the present disclosure, tannic acid, in various embodiments, can be incorporated in hydrogen bonded LbL films at physiologic pH.

Prior to building thin films, quartz crystal microbalance (QCM) was used to test whether interactions between thrombin and tannic acid exist at physiologic pH, and if so, whether they promote LbL assembly. Furthermore, to examine whether the mannitol excipient within the thrombin formulation (approximately 88% mannitol and 12% thrombin) affected potential film growth or was incorporated in the films, H-bond assembly under the same conditions was examined directly between mannitol and tannic acid. The two film architectures attempted were (thrombin/tannic acid)ₙ and (mannitol/tannic acid)ₙ, with a maximum of n=5 (where n is the number of bilayers) on an initial branched polyethyleneimine (BPEI) monolayer. The resulting frequency change for each architecture at a single harmonic is shown in FIG. 8A; decreasing frequency represents mass deposition. For (thrombin/tannic acid)ₙ, there is significant adsorption of thrombin and tannic acid at each respective deposition step, with some material desorption (increased frequency) during each rinse, signifying removal of non-specifically bound material. The overall decrease in frequency following each subsequent bilayer supports the formation of favorable hydrogen bonding interactions between tannic acid and thrombin at this deposition condition. In contrast, (mannitol/tannic acid)ₙ assembly does not occur. Although there is a small overall decrease in frequency, there is no drop in frequency at each mannitol deposition step, indicating no mannitol adsorption. In fact, following the first tannic acid deposition, each mannitol step acts like a rinse, removing some of the bound tannic acid. Like tannic acid, mannitol consists primarily of strong hydrogen bond donors and hydrogen bond acceptors in the form of hydroxyl groups. Unlike thrombin, however, mannitol lacks the multivalent and macromolecular structure typically needed for LbL film growth, thus disabling (mannitol/tannic acid)ₙ film assembly.

Having confirmed that a (thrombin/tannic acid)ₙ film could be successfully built at pH 7.4, lacking interference from the mannitol excipient, we explored the use of spray LbL to assemble these films. In this rapid LbL assembly technique, film components are aerosolized and sprayed at a substrate, leading to rapid multilayer formation. FIG. 8B shows the thickness of (thrombin/tannic acid)ₙ films sprayed on a monolayer of BPEI, for n=10, 25, and 50. Film thickness per bilayer is also shown. With increasing number of layers, film thickness per bilayer decreases significantly, transitioning from approximately 11 to 5 nm/bilayer over 10 to 25 bilayers, and from 5 to 2 nm/bilayer from 25 to 50 bilayers. This decrease in film thickness per bilayer may be a result of the significantly smaller size of tannic acid (1.7 kDa) compared to the large thrombin protein (approximately 36 kDa). With an increasing number of deposition steps, tannic acid may diffuse into the underlying film. This interdiffusion can alter film architecture, promoting less thrombin adsorption at increasing bilayer numbers, leading to a smaller increase in dry film thickness. Additionally, decrease in thickness per bilayer may be due to incomplete reversal of hydrogen bonding functionality following each deposition step.

Decrease in thrombin adsorption at increasing bilayers was also observed in the QCM growth of (thrombin/tannic acid)ₙ at just 5 bilayers as shown in FIG. 8A. The initial bilayer led to a frequency drop of approximately 140 Hz, corresponding to the total mass of thrombin and tannic acid adsorbed during the first bilayer. If an equivalent frequency drop were seen for the subsequent 4 bilayers, a final frequency change of approximately 700 Hz would be expected (not including initial BPEI deposition and wash). However, a total frequency drop of approximately 630 Hz was actually observed. Traditional dipped LbL assembly involves aqueous adsorption steps lasting several minutes, which can lead to rapid interdiffusion of small molecule components within these films. Therefore, dipped (thrombin/tannic acid)ₙ films may be expected to show a more dramatic decrease in film growth than what is observed for rapidly assembled sprayed films, in which the kinetics of interdiffusion may become rate-limited.

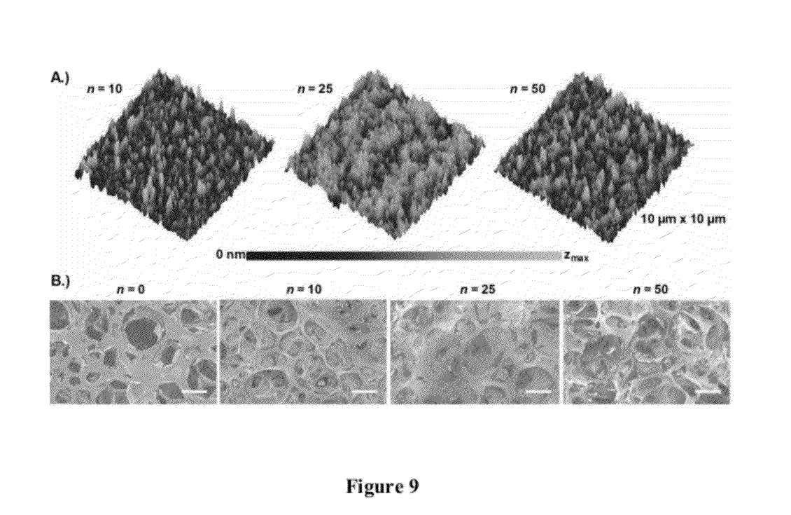

FIG. 9A shows the morphology of sprayed (thrombin/tannic acid)ₙ films measured via atomic force microscopy at n=10, 25, and 50. In general, the root-mean squared (rms) roughness (summarized in the figure caption), was found to increase with increasing film thickness. The rms roughness values were approximately 28% to 40% of the final film thickness, increasing with film thickness and ranging from 46.3±3.7 to 66.8±11.5 nm. In general, these films are rougher than typical spray LbL films that have roughness values in the range of just a few nanometers at greater than 100 bilayers. Previously reported dipped hydrogen bonded LbL films of block copolymer micelles and tannic acid, had rms roughness values of approximately 40% of the final film thickness (21 nm for a 50 nm thick film), which is comparable to the films developed here. Our films are comprised solely of a protein and a small molecule, in contrast to LbL films that contain polymeric components, which may be the reason for the large roughness values we observe.

The dissolution of sprayed (thrombin/tannic acid)ₙ films was examined after assembly on flat substrates for n=10, 25, and 50. FIG. 8C shows the change in film thickness over approximately 10 days in phosphate buffered saline (0.01 M PBS, 37° C.). This data was fit with a model for one-phase decay as described and reported in Table 51 in Supporting Information. The dissolution rate constant was calculated to be 8.2×10⁻³, 7.5×10⁻³, and 6.5×10⁻³ h⁻¹, for 10, 25, and 50 bilayer films, respectively. Dissolution half-life was calculated to be 84.8, 92.6, and 107.3 hours for 10, 25, and 50 bilayer films, respectively. There is an initial loss of approximately 25% to 47% of the film thickness in the first few hours of dissolution, followed by a more gradual decrease in film thickness over multiple days. This is expected, as there is no significant driving force for film deconstruction at these conditions (the same pH and ionic strength in which films were assembled); release is primarily diffusion based.

Having thoroughly characterized film properties on flat substrates, we used spray LbL assembly to coat a commercially available absorbent gelatin sponge with (thrombin/tannic acid)ₙ. In clinical use, this sponge is soaked in thrombin solution immediately prior to use. Direct incorporation of the hemostat as a coating within these sponges can provide a means of storing controlled amounts of thrombin in the sponge, allowing immediate administration in the operating room and on the battlefield, where standard clinical conditions are not available, and the ability to rapidly provide the hemostat can be life-saving. To promote film deposition throughout the 1 centimeter thick sponge, a vacuum was applied to the back of sponge during spray assembly. Plan-view scanning electron microscopy images of untreated and spray LbL coated sponges are shown in FIG. 9B. The thin film coating is visible on the sponge, but the underlying sponge architecture is completely maintained. The overall liquid absorption was observed for coated and uncoated sponges, and it was found that there was no change in PBS absorption between the coated sample and control, confirming that there is no significant change to the underlying sponge properties upon LbL coating.

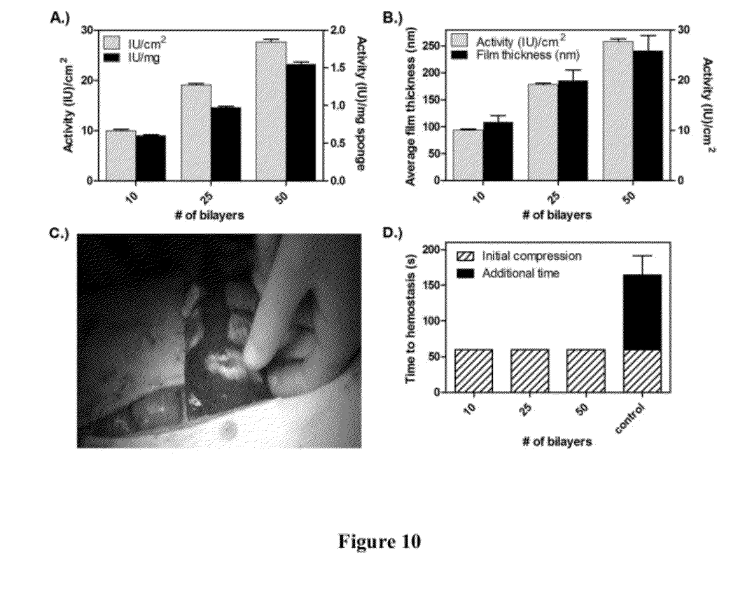

The activity of the thrombin LbL coated sponges was tested both in vitro and in vivo. In vitro activity was assessed by monitoring fibrin clot formation upon soaking coated sponges in solution and exposing this solution to fibrinogen (factor I), which is eventually converted to fibrin via the initial activity of thrombin. Film coated sponges were soaked over various times ranging from 10 minutes up to 6 days. No change in film activity was seen over this time period, which may be attributed to the possibility that most of the thrombin releases rapidly through diffusion from the multilayer film. This rapid release of thrombin may cause the large initial loss in film thickness that is seen on film coated flat substrates. FIG. 10A shows activity of coated sponges that were soaked for 10 minutes expressed as international units (IU) per milligram of sponge and IU per square centimeter of sponge. As expected, activity increases with increasing number of film bilayers. FIG. 10B simultaneously shows a plot of activity and film thickness; activity increases monotonically with increasing bilayer number and film thickness. The levels of in vitro activity quantified for n=10, 25, and 50 bilayers are each clinically relevant and significant, where a single IU of thrombin activity is known to clot 1 mL of plasma in 15 seconds.

A porcine spleen bleeding model, commonly used to test commercially available hemostatic products, was used to assess the in vivo activity of film coated sponges. Sponges coated with a monolayer of BPEI and uncoated gauze were also tested as controls. The porcine spleen was exposed and a wound was inflicted; bleeding intensity was classified and the test sample was applied with light pressure. FIG. 10C shows a representative image of this surgery, while the quantified results are shown in FIG. 10D. Sixty seconds of digital compression was always applied once the test sample was placed on the bleeding wound. As shown in FIG. 10D, none of the (thrombin/tannic acid)ₙ sponge formulations required additional time or compression to promote hemostasis following this initial compression period. In each case, bleeding stopped during the initial 60 second compression period. For the BPEI controls, an additional 104±27 seconds were needed to stop bleeding, including an additional 30 second compression period following the first compression. Uncoated cotton gauze controls did not promote any hemostasis over the 12 minute test period and are not represented in FIG. 10D. From in vitro assays, it is clear that there are greater amounts of thrombin in films with higher bilayer numbers. In vivo assays could not demonstrate differences in activity between these films, due to the standard 60 second compression period; however, all LbL film formulations provide sufficient thrombin to enable hemostasis in this time. Overall, all of the film coated sponges acted instantaneously following the initial compression, with greatly enhanced activity compared to a BPEI coated sponge control which requires additional compression and time to reach hemostasis. Therefore, the (thrombin/tannic acid)ₙ film architecture is highly promising in promoting hemostasis in a clinically relevant animal model at as few as 10 bilayers.

Here we have used the spray LbL assembly method to formulate a novel LBL film aimed at promoting hemostasis. In contrast to traditional multilayer films, this architecture does not contain any synthetic polymeric component. The exemplary LBL films were formulated based on interactions between natural components, thrombin and tannic acid, at physiologic pH. Each of these materials is FDA approved, making these films amenable to rapid clinical translation. We demonstrated the practical applicability of these films to a clinically relevant absorbent gelatin sponge and showed that film coated sponges were capable of leading to rapid hemostasis in a porcine spleen bleeding model.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A coated device comprising:
a substrate;
a film coating at least part of the substrate, which film comprises a multilayer unit comprising a first layer and a second layer associated with one another via a hydrogen bond, wherein the first layer comprises a first natural polymeric material and a hydrogen bond donor and wherein the second layer comprises a second natural polymeric material and a hydrogen bond acceptor; and
an agent for delivery associated with the coated device such that, decomposition of one or more layers of the film results in release of the agent.

2. The coated device of claim 1, wherein at least one of the first and second layer consists of or comprises the agent for delivery.

3. The coated device of claim 1, wherein the multilayer unit is selected from the group consisting of a bilayer, a trilayer and a tetralayer.

4. The coated device of claim 1, wherein the number of the multilayer unit is selected from the group consisting of 3, 5, 10, 20, 30, 40, 50, 60, 70, 80, 90, 100, 150 and 200.

5. The coated device of claim 1, wherein the multilayer unit is a bilayer.

6. The coated device of claim 1, wherein the hydrogen bond donor is a phenolic or amido group.

7. The coated device of claim 1, wherein the hydrogen bond acceptor is a carboxyl or sulfate group.

8. The coated device of claim 1, wherein the first layer consists of or comprises a polypeptide.

9. The coated device of claim 8, wherein the polypeptide is a clotting factor.

10. The coated device of claim 9, wherein the clotting factor is selected from the group consisting of fibrinogen, thrombin, tissue factor, von Willebrand factor, fletcher factor, fitzgerald factor, fibronectin, antithrombin III, heparin cofactor II, protein C, protein S, protein Z, ZPI, plasminogen, alpha 2-antiplasmin, tPA, urokinase, PAIL PAI2, cancer procoagulant, and fragments and variants thereof.

11. The coated device of claim 10, wherein the clotting factor is thrombin.

12. The coated device of claim 1, wherein the second layer consists of or comprises a polymeric acid.

13. The coated device of claim 1, wherein the second layer consists of or comprises a tannic acid.

14. The coated device of claim 1, further comprising a base layer.

15. The coated device of claim 1, wherein the base layer consists of or comprises polyethyleneimine (PEI).

16. The coated device of claim 1, further comprising a second multilayer unit, wherein the second multilayer comprises a polyelectrolyte.

17. The coated device of claim 1, wherein the substrate is a medical device.

18. The coated device of claim 17, wherein the medical device is selected from the group consisting of stents, catheters, balloons, guide wires, grafts, artificial vessels, artificial valves, filters, vascular closure systems, shunts, artificial ligaments and prosthetics.

19. A method of using a coated device comprising:
contacting/implanting in or on a body the coated device comprising
a substrate;
a film coating at least part of the substrate, which film comprises a multilayer unit comprising a first layer and a second layer associated with one another via a hydrogen bond, wherein the first layer comprises a first natural polymeric material and a hydrogen bond donor and wherein the second layer comprises a second natural polymeric material and a hydrogen bond acceptor; and
an agent for delivery associated with the coated device such that, decomposition of one or more layers of the film results in release of the agent; and
releasing the agent for delivery.

20. A method of assembling a film layer-by-layer on at least part of substrate comprising:
depositing the film on the at least part of the substrate, wherein the film comprises
a multilayer unit comprising a first layer and a second layer associated with one another via a hydrogen bond,
wherein the first layer comprises a first natural polymeric material and a hydrogen bond donor and wherein the second layer comprises a second natural polymeric material and a hydrogen bond acceptor; and
associating, prior to, during or after the step of depositing, an agent for delivery with the coated device such that, decomposition of one or more layers of the film results in release of the agent.

21. The method of claim 20, wherein the step of depositing is carried out at around physiologic pH.

22. The method of claim 20, wherein the step of depositing comprises spraying the first layer and the second layer alternatively.

23. The method of claim 20, wherein the step of spraying is performed under vacuum.

24. The method of claim 20, wherein the step of spraying is performed under vacuum of about 50 psi.

25. The method of claim 20, further comprising depositing a base layer on the substrate before the step of spraying.

26. The method of claim 20, further comprising plasma etching the substrate before the step of spraying.

27. A coated device comprising:
a substrate;
a film coating at least part of the substrate, which film comprises a multilayer unit comprising a tetralayer with alternating layers of opposite charge; and
an agent for delivery associated with the coated device such that, decomposition of one or more layers of the film results in release of the agent.

28. The coated device of claim 27, wherein the agent is a small molecule.

29. (canceled)

30. The coated device of claim 27, wherein at least one layer of the tetralayer consists of or comprises a polyelectrolyte selected from the group consisting of polyesters, polyanhydrides, polyorthoesters, polyphosphazenes, polyphosphoesters, and any combinations thereof.

31. (canceled)

32. (canceled)

33. The coated device of claim 27, wherein the agent for delivery consists of or comprises an therapeutic agent.

34. The coated device of claim 33, wherein the therapeutic agent is selected from the group consisting of an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, and angiogenesis inhibitor.

35. The coated device of claim 34, wherein the therapeutic agent is an antibiotics.

36. The coated device of claim 35, wherein the antibiotic is selected from the group consisting of β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim.

37. The coated device of claim 36, wherein the antibiotic is vancomycin.

38. A method of using a coated device comprising:
contacting/implanting in or on a body the coated device comprising a substrate;
a film coating at least part of the substrate, which film comprises a multilayer unit comprising a tetralayer with alternating layers of opposite charge; and
an agent for delivery associated with the coated device such that, decomposition of one or more layers of the film results in release of the agent; and
releasing the agent for delivery.

39. A method of assembling a film layer-by-layer on at least part of substrate comprising:
depositing the film on the at least part of the substrate, wherein the film comprises
a substrate;
a film coating at least part of the substrate, which film comprises a multilayer unit comprising a tetralayer with alternating layers of opposite charge; and
associating, prior to, during or after the step of depositing, an agent for delivery with the coated device such that, decomposition of one or more layers of the film results in release of the agent.
